(19) United States
(12) Patent Application Publication
(10) Pub. No.: US 2023/0038960 A1
(43) Pub. Date: Feb. 9, 2023

(54) PHOTOCATALYTIC DEVICE AND METHOD OF MANUFACTURING PHOTOCATALYTIC DEVICE

(71) Applicant: NIKKISO CO., LTD., Tokyo (JP)

(72) Inventors: Ikuo MITO, Tokyo (JP); Takeshi MIYAZATO, Tokyo (JP)

(21) Appl. No.: 17/880,245

(22) Filed: Aug. 3, 2022

Related U.S. Application Data
(63) Continuation of application No. PCT/JP2021/002525, filed on Jan. 26, 2021.

(30) Foreign Application Priority Data
Feb. 4, 2020 (JP) 2020-017017

(51) Int. Cl. A61L 9/20; B01J 23/20; B01J 35/00; B01J 37/08; B01J 37/14

(52) U.S. Cl. CPC A61L 9/205; B01J 23/20; B01J 35/004; B01J 37/08; B01J 37/14; A61L 2209/12

(57) ABSTRACT

A photocatalytic device includes: a metal layer; and a photocatalytic layer provided on the metal layer and containing a photocatalytic material. In the photocatalytic layer, a slit or an opening is formed to expose a portion of the metal layer.

### RELATED APPLICATION

This application is a continuation application of International Application No. PCT/JP2021/002525, filed Jan. 26, 2021, which claims priority to Japanese Patent Application No. 2020-017017, filed Feb. 4, 2020. The entire contents of all of these applications are incorporated herein by reference.

### BACKGROUND OF THE INVENTION

### 1. Field of the Invention

The present invention relates to photocatalytic devices.

### 2. Description of the Related Art

Photocatalytic substances that absorb light and exhibit a catalytic action are known. By impinging ultraviolet light, visible light, etc. on a photocatalytic substance, a chemical reaction that is difficult in an ordinary catalytic process can be induced at an ordinary temperature. A photocatalyst promotes an oxidation-reduction reaction and so is capable of decomposing organic substances and bacteria. Various photocatalytic devices are invented. Recently, methods of spatially isolating excited electrons from holes are employed in order to suppress recombination of excited electrons and holes formed by light irradiation and to make the oxidation-reduction reaction induced by the photocatalyst more efficient. These methods are designed to isolate excited electrons from holes, by causing the photocatalyst to carry a metal to let excited electrons flow into the metal or by configuring the photocatalyst as a heterostructure comprised of a plurality of layers having different band structures.

For example, a method of forming a photocatalytic layer of a titanium oxide on a quartz substrate and forming thereon a patterned deposited film of silver, using closely arranged polystyrene microspheres as a mask, is invented (for example, CN-100505333). Also, a photocatalyst including a first copper oxide layer formed on top of a transparent substrate, a second copper oxide layer formed on top of the first copper oxide layer, and a zinc oxide layer formed on top of the second copper oxide layer (for example, KR Patent 10-179708) is invented.

The photocatalytic devices mentioned above are structured to isolate excited electrons from holes by using an Ag deposited film/titanium oxide layer structure or a zinc oxide layer/copper oxide layer structure. However, the Ag deposited film pattern has a disadvantage in that excited electrons travel a long distance, i.e., several μm, before arriving at the Ag deposited film as a support electrode and are recombined with holes in the middle. Further, the resistance of copper oxide is generally very large, i.e., from 10⁵ Ωm (thick film) to 10¹⁰ Ωm (thin film), and it is difficult to use holes flowing from the zinc oxide layer into the copper oxide layer as an electric current. For this reason, the efficiency of producing substances like hydroxy radicals and superoxide anions, which are important for oxidation-reduction and decomposition of organic substances, bacteria, etc. will be poor.

### SUMMARY OF THE INVENTION

The present invention addresses the above-described issues, and an illustrative purpose thereof is to provide a novel technology capable of decomposing organic substances, bacterial, etc. efficiently and, for example, to provide a novel photocatalytic device that suppresses recombination of electrons excited in the catalytic layer with holes.

A photocatalytic device according to an embodiment of the present invention includes: a metal layer; and a photocatalytic layer provided on the metal layer and containing a photocatalytic material. In the photocatalytic layer, a slit or an opening is formed to expose a portion of the metal layer.

A photocatalytic device according to another embodiment of the present invention includes: a metal layer made of an SUS (Steel Use Stainless) material; and a photocatalytic layer provided on the metal layer and containing a tantalum oxide.

Another embodiment of the present invention relates to a method of manufacturing a photocatalytic device. The method includes heating and calcinating an SUS substrate coated with a tantalum-containing agent in an oxygen-containing atmosphere at 700° C. or above, at which the SUS substrate is not melted, to form a photocatalytic layer containing a tantalum oxide on the SUS substrate.

Optional combinations of the aforementioned constituting elements, and implementations of the invention in the form of methods, apparatuses, and systems may also be practiced as additional modes of the present invention.

### BRIEF DESCRIPTION OF THE DRAWINGS

FIG. 1 is a perspective view showing a schematic configuration of a photocatalytic device according to the first embodiment;

FIG. 2 is a top view showing an important part of the photocatalytic device according to the first embodiment;

FIG. 3 is a schematic diagram illustrating a mechanism of decomposition of an attached substance in the photocatalytic device according to the first embodiment;

FIG. 4 is a cross-sectional view schematically showing an A-A cross section of FIG. 2;

FIG. 5 is a perspective view of a photocatalytic device according to the second embodiment;

FIG. 6 is a top view showing an important part of the photocatalytic device according to the second embodiment;

FIG. 7 is a cross-sectional view showing an important part of a photocatalytic device according to a variation of the first embodiment;

FIG. 8 is a schematic diagram illustrating a method of manufacturing a photocatalytic device according to the third embodiment;

FIGS. 9A-9C are graphs showing X-ray diffraction patterns for illustrating the relationship between the pull-up speed of the SUS430 substrate and the crystalline nature of the tantalum oxide film formed on the substrate by calcination; and

FIG. 10A shows an SEM image of the SUS430 substrate surface without a coating film, FIG. 10B is an SEM image of the SUS430 substrate surface on which a coating film is formed at the pull-up speed of 2 cm/min, FIG. 10C shows an SEM image of the SUS430 substrate surface on which a coating film is formed at the pull-up speed of 10 cm/min, and FIG. 10D is an SEM image of the SUS430 substrate surface on which a coating film is formed at the pull-up speed of 20 cm/min.

### DETAILED DESCRIPTION OF THE INVENTION

The invention will now be described by reference to the preferred embodiments. This does not intend to limit the scope of the present invention, but to exemplify the invention.

The photocatalytic device according to an embodiment of the present disclosure is provided with a metal layer and a photocatalytic layer provided on the metal layer and containing a photocatalytic material. In the photocatalytic layer, a slit or an opening is formed to expose a portion of the metal layer. According to this embodiment, recombination of electrons and holes produced in the photocatalytic layer is suppressed because electrons or holes produced in the photocatalytic layer enter the metal layer. Further, when organic substances, bacteria, etc. subject to decomposition come into contact with both the photocatalytic layer and the metal layer, contact between the target of decomposition and the metal layer produces radical-induced decomposition intervened by electrons, and contact between the target of decomposition and the photocatalytic layer produces radical-induced decomposition intervened by holes. The decomposition reaction is promoted in the photocatalytic device as a whole.

The width of the slit or the inner diameter of the opening may be in a range of 1-30 μm. More preferably, the width or the inner diameter may be in a range of 1-5 μm. This facilitates contact of bacteria and allergens with the metal layer as well as the photocatalytic layer.

The photocatalytic layer may be configured such that a plurality of cells are arranged to sandwich the slit. This allows organic substances, bacteria, etc. attached to the cells to be decomposed individually.

The photocatalytic material may be an oxide of the metal element included in the metal layer. This makes it possible to configure a portion of the metal layer as a photocatalytic layer.

The photocatalytic layer may be produced by anode oxidation of a portion of the metal layer. This allows obtaining a photocatalytic layer including a photocatalytic material formed to have a large surface area (e.g., a minute columnar body).

The photocatalytic material may be at least one compound selected from the group consisting of titanium oxide, tantalum oxide, cadmium sulfide, and zinc oxide.

The metal layer may be made of a material containing at least one element selected from the group consisting of tantalum, titanium, and aluminum. Alternatively, the metal layer may be made of an SUS material.

The photocatalytic material may be a material in which a photocatalytic reaction is induced by ultraviolet light having a peak wavelength in a range of 250-300 nm. The photocatalytic material may be a material in which a photocatalytic reaction is induced by ultraviolet light having a peak wavelength in a range of 270-290 nm. In this way, a photocatalytic material having a strong oxidation power and a reduction power can be used.

The photocatalytic device may further include an ultraviolet irradiation part that irradiates the photocatalytic layer with ultraviolet light. The ultraviolet irradiation part may include an LED that radiates an ultraviolet light having a peak wavelength in a range of 250-300 nm. By irradiating the photocatalytic material having a strong oxidation power and a reduction power with the ultraviolet light emitted by the LED, a photocatalytic device that promotes a photocatalytic reaction can be realized.

The metal layer may be configured such that a positive voltage is applied while the photocatalytic layer is irradiated with an ultraviolet light. This facilitates adhesion of generally negatively charged bacteria, etc. and improves the decomposition efficiency.

A photocatalytic device according to another embodiment of the present disclosure is provided with a metal layer made of an SUS material and a photocatalytic layer provided on the metal layer and containing a tantalum oxide. According to this embodiment, organic substances, bacteria, etc. can be decomposed efficiently.

Another embodiment of the present disclosure relates to a method of manufacturing a photocatalytic device. The method includes heating and calcinating an SUS (Steel Use Stainless) substrate coated with a tantalum-containing agent in an oxygen-containing atmosphere at 700° C. or above, at which the SUS substrate is not melted, to form a photocatalytic layer containing a tantalum oxide on the SUS substrate. According to this embodiment, the use of an SUS substrate that is not melted at a relatively high temperature allows calcination at a high temperature and formation of a photocatalytic layer containing a tantalum oxide having a favorable crystalline nature. This can improve the efficiency of decomposition of organic substances, bacteria, etc. by the photocatalytic device.

A description will be given of embodiments of the present invention with reference to the drawings. Like numerals represent like elements so that the description will be omitted accordingly. The relative dimensions of the constituting elements in the drawings do not necessarily mirror the relative dimensions in the actual photocatalytic device. The structure described below is by way of example only and does not limit the scope of the invention.

### First Embodiment

### (Photocatalytic Device)

Recently, air cleaners configured to decompose bacteria, fungus, and allergic substances by irradiating a photocatalyst with an ultraviolet light have been developed. An issue associated with using a photocatalyst is that (a) electrons excited by light irradiation of the photocatalyst and holes are spatially collocated and so are recombined promptly. Therefore, the proportion of electrons and holes used for water decomposition and oxygen decomposition outside the photocatalyst is small. Consequently, the efficiency of producing hydroxy radicals and superoxide anions for oxidation and reduction or hazardous chemical substances will be low. Meanwhile, it is also important to (a) ensure a large absorption coefficient of the photocatalyst and (c) ensure a large surface area to facilitate an oxidation and reduction reaction.

In light of the foregoing discusses, the inventors have focused on the following issues.

(a) It is preferable to isolate excited electrons and holes spatially in order to suppress recombination of excited electrons and holes. For example, excited electrons and holes may be isolated by, for example, causing the photocatalyst to carry a metal to let excited electrons flow into the metal or by configuring a so-called heterostructure in which the photocatalyst is connected to a plurality of layers having different band structures.

(b) It is preferable to excite electrons by a light having a wavelength close to the absorption peak of the photocatalytic substance in order to increase the efficiency of light absorption by the photocatalyst.

(c) It is preferable to secure a large area of the photocatalyst by using a nano-sized photocatalytic substance, in order to promote a chemical reaction on the photocatalyst surface.

In this background, we have invented a photocatalytic device illustrated below, in which a photocatalyst is formed on a metal substrate. In this photocatalyst device, the photocatalyst layer is divided into microcells. By allowing attached organic substances, etc. to be fully in contact with the photocatalyst layer containing holes and the metal layer containing electrons, a decomposition reaction induced by the photocatalyst is promoted. Further, a photocatalytic reaction is promoted by giving an electric bias to the electrode layer (metal layer).

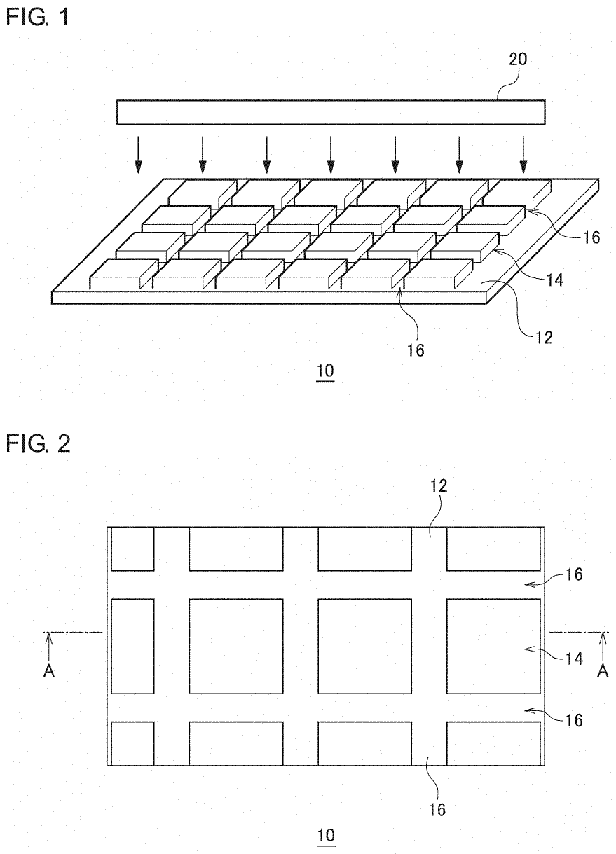

FIG. 1 is a perspective view showing a schematic configuration of a photo catalytic device according to the first embodiment. FIG. 2 is a top view showing an important part of the photocatalytic device according to the first embodiment. A photocatalytic device 10 is provided with a plate-shaped metal layer 12 and a photocatalytic layer 14 provided on the metal layer 12 and containing a photocatalytic material. A slit 16 is formed in the photocatalytic layer 14 to expose a portion of the metal layer 12, and a plurality of microcells 14a are arranged vertically and horizontally, sandwiching the slit 16. The thickness of the photocatalytic layer 14 is about 50-200 nm.

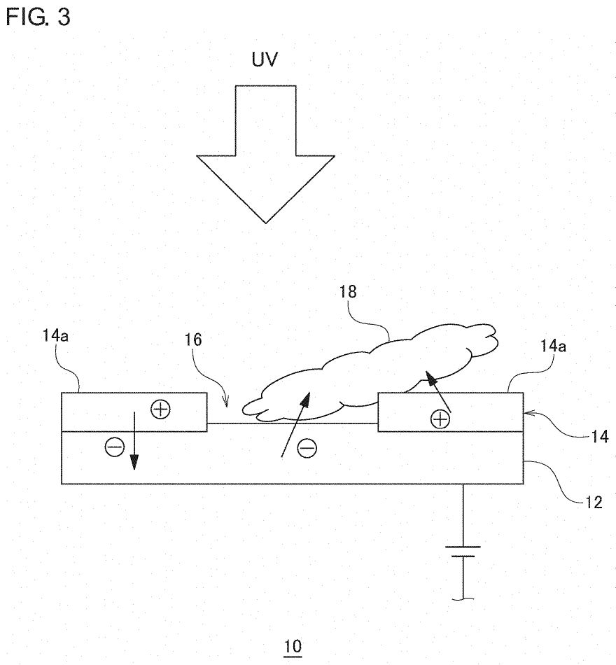

FIG. 3 is a schematic diagram illustrating a mechanism of decomposition of an attached substance in the photocatalytic device according to the first embodiment. As shown in FIG. 3, excited electrons and holes that remain are produced in the microcell 14a irradiated with an ultraviolet light. If the microcell 14a is an isolated system, electrons and holes are recombined immediately, lowering the efficiency of production of radicals that are important for oxidation and reduction by a photocatalytic action.

In the photocatalytic device 10, however, electrons or holes produced in the microcell 14a are caused to flow into the metal layer 12. Thereby, electrons and holes can be isolated spatially to suppress recombination of electrons and holes produced in the microcell 14a. Consequently, each of electrons and holes can be used effectively to produce radicals. In this embodiment, a positive bias voltage is applied to the metal layer 12. Consequently, electrons excited in the microcell 14a are attracted by the metal layer 12 characterized by an electric(al) conductivity higher than that of the photocatalytic layer 14, which is made of a metallic oxide, and by a high mobility. The metal layer 12 is in contact with the entirety of the bottom surface of the photocatalytic layer 14 so that excited electrons flow into the metal layer 12 promptly. Further, by configuring the metal layer 12 such that a positive voltage is applied while the photocatalytic layer 14 is being irradiated with an ultraviolet light, adsorption of generally negatively charged bacteria, etc. is facilitated, and the decomposition efficiency is improved.

Further, an attached substance 18 such as organic substances and bacteria subject to decomposition can be in contact with both the photocatalytic layer 14 and the metal layer 12 in the photocatalytic device 10. Therefore, contact between the attached substance 18 and the metal layer 12 produces decomposition induced by radicals such as superoxide anions and intervened by electrons, and contact between the attached substance 18 and the microcell 14a produces decomposition induced by hydroxy radicals and intervened by holes. The decomposition reaction is promoted in the photocatalytic device as a whole.

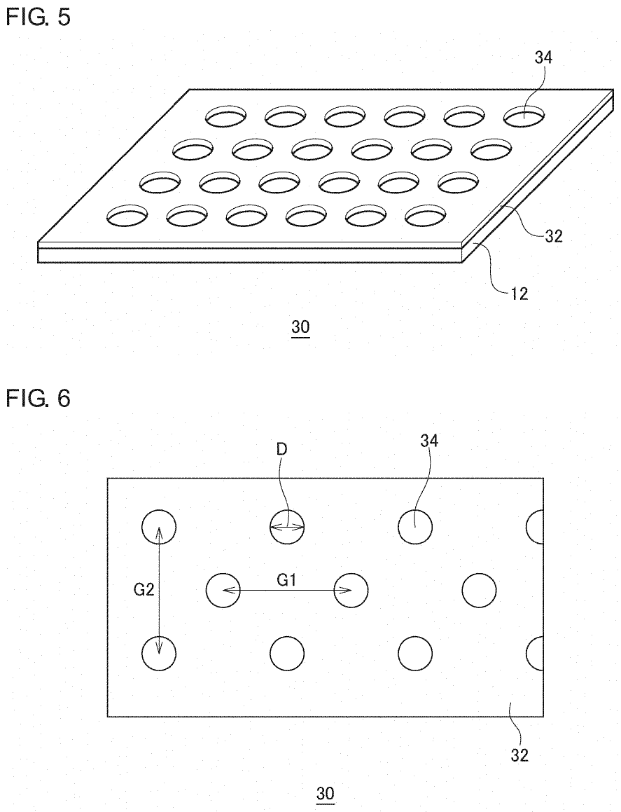

It is preferable that the resistance of the metal layer 12 according to this embodiment is in a range of 10⁻⁴ Ωm-10⁻³ Ωm, which is extremely smaller than that of a metal oxide layer. Electrons or holes flowing into the metal layer 12 having such a low resistance move in the metal layer 12 and easily arrive at the slit 16 or the opening 34 in which the metal layer 12 is exposed (see FIG. 5).

### (Photocatalytic Material)

A description will now be given of the photocatalytic material included in the photocatalytic layer 14. The photocatalytic material is not limited to any particular material so long as it achieves a photocatalytic action. For example, a compound such as titanium oxide, tantalum oxide, cadmium sulfide, and zinc oxide may be used. In consideration of the fact that the photocatalytic layer 14 is formed on the metal layer 12, an oxide of a metal element included in the metal layer 12 may be used. This makes it possible to selectively configure a portion of the metal layer 12 as the photocatalytic layer 14 by an existent method such as oxidation process or anode oxidation used in manufacturing of a semiconductor. According to the method that uses anode oxidation, in particular, the photocatalytic layer 14 of a form having a large surface. For example, the photocatalytic layer 14 having a nano-sized minute columnar body (nanotube structure)) is obtained.

In the case the metal layer 12 is made of tantalum, for example, a tantalum oxide (Ta₂O₅) can be used in combination to produce the photocatalytic layer 14. Further, in the case the metal layer 12 is made of titanium, a titanium oxide (TiO₂) can be used in combination to produce the photocatalytic layer 14. Alternatively, aluminum (Al), which is characterized by a high ultraviolet reflectivity, may be used in the metal layer 12 in order to reflect the ultraviolet light transmitted through the photocatalytic layer 14 on the surface of the metal layer 12 for use in the photocatalytic process in the photocatalytic layer 14 again.

### (Tantalum Oxide as a Photocatalytic Substance)

Titanium oxide, discussed above, is suitable as a photocatalyst excited by an ultraviolet light (UVA range). The potential energy of excited electrons and holes involved in an oxidation-reduction action is determined by the energy levels of the conduction band and the valance band and by the bandgap energy representing a difference in the energy levels of the conduction band and the valence band. The bandgap energy of titanium oxide (TiO₂) is 3.2 eV, which translates into a wavelength of 387 nm so that the energy in the UVA range defines a limit. We have focused on tantalum oxide (Ta₂O₅: bandgap energy of 4.0 eV (translating into a wavelength of 310 nm)), which is capable of providing electrons and holes with a larger potential energy, and we have arrived at an idea of using deep ultraviolet LED (peak wavelength of 280 nm: translating into an energy of 4.4 eV), which fully exploits the characteristics of tantalum oxide.

Thus, the photocatalytic material may be a material in which a photocatalytic reaction is induced by an ultraviolet light having a peak wavelength in a range of 250-300 nm. More preferably, the photocatalytic material may be a material in which a photocatalytic reaction is induced by an ultraviolet light having a peak wavelength in a range of 270-290 nm. Thus, a photocatalytic material having a strong oxidation power and reduction power can be used.

The photocatalytic device 10 according to this embodiment is further provided with an ultraviolet irradiation unit 20 adapted to irradiate the photocatalytic layer 14 with an ultraviolet light. The ultraviolet irradiation unit 20 includes an LED that radiates an ultraviolet light having a peak wavelength in a range of 250-300 nm. By irradiating the photocatalytic layer 14 including a photocatalytic material having a strong oxidation power and reduction power with the ultraviolet light emitted by the LED, a photocatalytic device that promotes a photocatalytic reaction can be realized.

### (Slit Size)

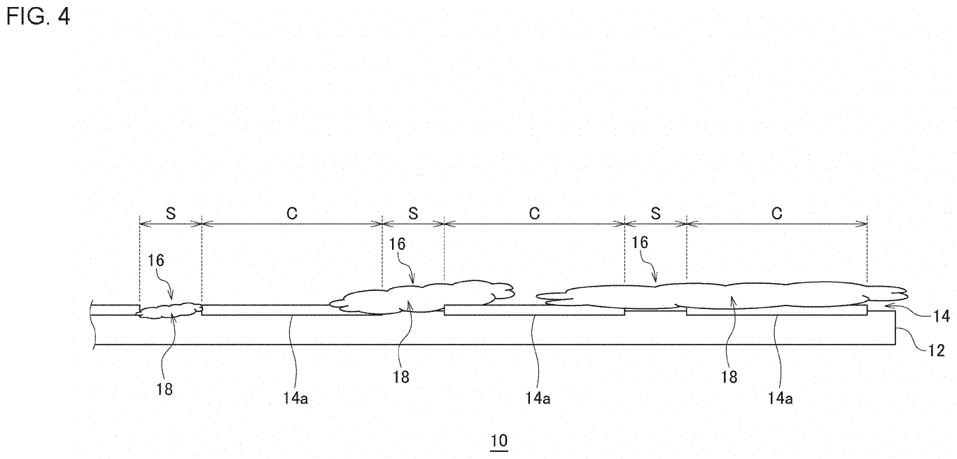

The size of bacteria and hazardous chemical substances such as formaldehyde ranges very extensively, namely, 1 nm-several μm. For this reason, it is difficult to set a slit (opening) optimum for chemical substances of all sizes in the metal layer 12. The size of a slit suitable for the photocatalytic device 10 targeting bacteria and allergens will be discussed. FIG. 4 is a cross-sectional view schematically showing an A-A cross section of FIG. 2. FIG. 4 shows a relationship between a size C of one side of the microcell 14a and a width S of the slit 16. The figure illustrates how the attached substance 18 such as bacteria is attached to the metal layer 12 and the photocatalytic layer 14.

The size C of one side of the microcell 14a is, for example, about 1-100 μm. The size C of one side is, more preferably, 3-10 μm. The width S of the slit 16 in the photocatalytic layer 14 is, for example, about 0.5-30 μm. The width S of the slit may be in a range of 1-30 μm. More preferably, the width S is in a range of 1-5 μm. This makes it easy for bacteria or allergens to be in contact with the metal layer 12 as well as with the photocatalytic layer 14.

### Second Embodiment

FIG. 5 is a perspective view of a photocatalytic device according to the second embodiment. FIG. 6 is a top view showing an important part of the photocatalytic device according to the second embodiment. A photocatalytic device 30 is mainly characterized in that a photocatalytic layer 32, in which a plurality of openings 34 are formed in one continuous layer, is provided on the metal layer 12. A description of features and advantages similar to those of the first embodiment will be omitted.

Intervals G1, G2 between the openings are, for example, about 1-100 μm. An inner diameter D of the opening 34 is, for example, about 0.5-30 μm. The inner diameter D of the opening 34 may be in a range of 1-30 μm. More preferably, the inner diameter D is in a range of 1-5 μm. This makes it easy for bacteria or allergens to come into contact with the metal layer 12 as well as the photocatalytic layer 14.

### (Variation 1)

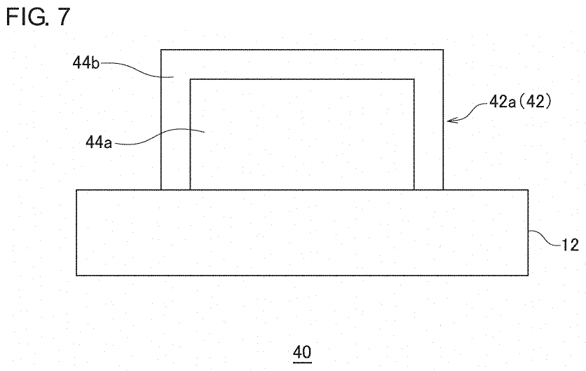

FIG. 7 is a cross-sectional view showing an important part of a photocatalytic device according to a variation of the first embodiment. In a photocatalytic device 40 shown in FIG. 7, a microcell 42s of a photocatalytic layer 42 has a two-layers structure. In the case the metal layer 12 is made of tantalum, the microcell 42a includes a first layer 44a made of a tantalum oxide and a second layer 44b made of a titanium oxide and covering the first layer 44a. By stacking a plurality of materials with different energy band structures, recombination of excited electrons and holes can be suppressed more successfully.

### Third Embodiment

### (Method of Manufacturing a Photocatalytic Device)

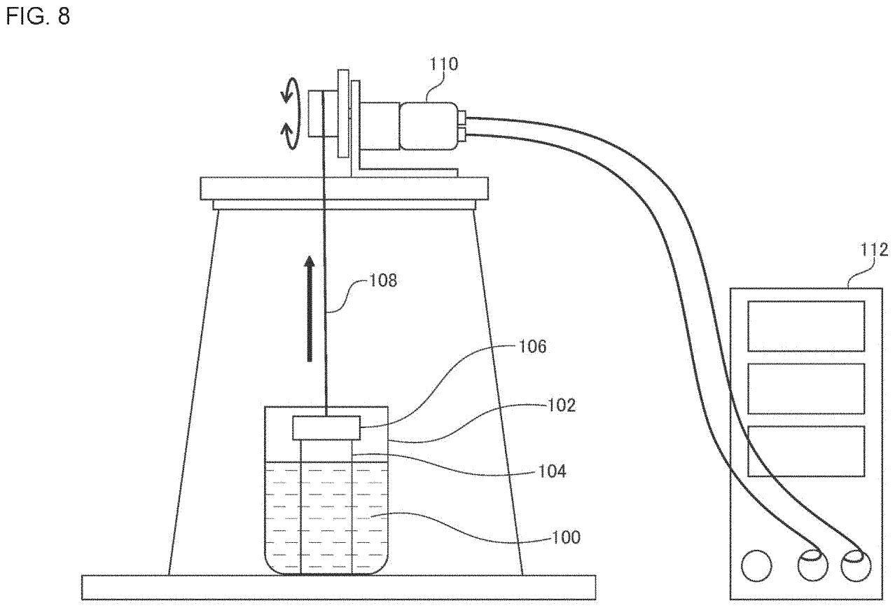

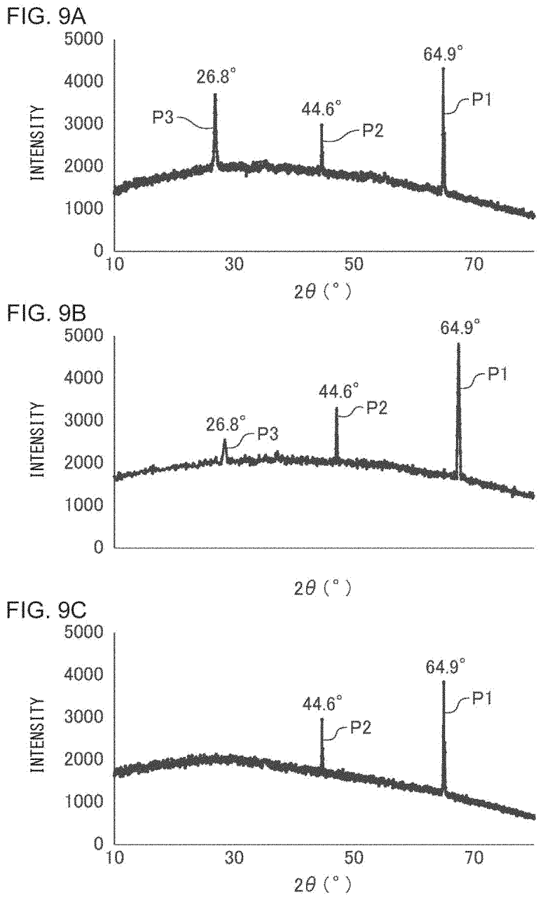

FIG. 8 is a schematic diagram illustrating a method of manufacturing a photocatalytic device according to the third embodiment. The manufacturing method according to this embodiment uses the metal organic decomposition (MOD) method, but the method is not limited to this.

In this embodiment, dip coat-precursors are used in the MOD method. Ta-10-P (Ta₂O₅ coating material: manufactured by Kojundo Chemical Lab. Co., Ltd.) is used as a dip coat-precursor. A coating liquid 100 adjusted such that the concentration of the coating material with respect to n-butyl acetate as a solvent is 10% is prepared. The coating liquid 100 is then poured into a beaker 102 until the coating liquid 100 reaches a predetermined depth.

Various substrates necessary for preparation of samples are then made available. Samples are prepared by using five types of substrates, namely, SUS430, SUS316, tantalum (Ta), copper (Cu), and aluminum (Al) substrates. As shown in FIG. 8, one end of a substrate 104 is retained by a clip 106. One end of a wire 108 tied to the clip 106 is wound around the rotation shaft of a motor 110. In this state, the position of the clip 106 is adjusted so that the majority of the substrate 104 is immersed in the coating liquid 100.

The driving voltage of the motor 110 is adjusted by a DC driving power source 112 to pull up the substrate 104 from the coating liquid 100 at a predetermined speed. More specifically, the substrate is pulled up at a speed of 2-20 cm/min. The higher the pull-up speed, the thicker the coating of the coating liquid 100 on the substrate 104 pulled up tends to be.

The substrate 104 coated with the coating liquid 100 is maintained for 10 minutes in an air atmosphere of 150° C. in a furnace. After the coating film is dried, the substrate 104 is cooled to an ordinary temperature. The substrate 104 is then heated to 430° C. at a rate of temperature increase of 20° C./min in a furnace in an air atmosphere and maintained for 15 minutes. The substrate 104 is then heated to a desired calculation temperature at a rate of temperature increase of 7° C./min and maintained for 30 minutes. The substrate 104 is then cooled to 100° C. at a rate of temperature decrease of 7° C./min, taken out of the furnace, and cooled slowly.

Table 1 shows results of observation of the coating film on photocatalytic device samples comprised of the respective substrates prepared by the manufacturing method described above.

TABLE 1

| | MELTING | | | | | |
|---|---|---|---|---|---|---|
| SUBSTRATE | POINT | | | | | |
| MATERIAL | (° C.) | | | | | |
| SUS430 | 1400-1450 | — | — | OK | OK | OK |
| | | | | (AMORPHOUS) | (CRYSTALLIZED) | (PROPERLY |
| | | | | | | CRYSTALLIZED) |
| SUS316 | 1370-1400 | OK | OK | NG | — | — |
| | | (AMORPHOUS) | (AMORPHOUS) | (SILVER-WHITE | | |
| | | | | OXIDATION) | | |
| Ta | 3017 | OK | OK | NG | NG | — |
| | | (AMORPHOUS) | (AMORPHOUS) | (WHITE | (WHITE | |
| | | | | OXIDATION) | OXIDATION) | |
| Cu | 1085 | NG | — | — | — | — |
| | | (BLACK | | | | |
| | | OXIDATION) | | | | |
| Al | 660 | OK | OK | — | — | — |
| | | (AMORPHOUS) | (AMORPHOUS) | | | |
| | | 500 | 550 | 700 | 800 | 900 |

CALCINATION TEMPERATURE (° C.)

In the case the substrate is made of aluminum, the surface condition of both substrates calcinated at 500° C., 550° C. is proper, but the coating film is in an amorphous state. Further, the substrate calcinated at 550° C. is softened. In the case the substrate is made of copper, the substrate calcinated at 500° C. is softened, and the surface is oxidized and turned black. In the case the substrate is made of tantalum, the surface condition of both substrates calcinated at 500° C., 550° C. is proper, but the coating film is in an amorphous state. Further, the surface of the substrates calcinated at 700° C., 800° C. is oxidized and turned white.

In the case the substrate is made of SU316, the surface condition of both substrates calcinated at 500° C., 550° C. is proper, but the coating film is in an amorphous state. Further, the surface of the substrates calcinated at 700° C. is oxidized and turned silverish white. In the case the substrate is made SU430, weak crystallization is observed at the calcination temperature of 800° C. At the calcination temperature of 900° C., the SU430 substrate is not softened, the surface condition is proper, and proper crystals with aligned crystal orientations are formed on the surface.

FIGS. 9A-9C are graphs showing X-ray diffraction patterns for illustrating the relationship between the pull-up speed of the SUS430 substrate and the crystalline nature of the tantalum oxide film formed on the substrate by calcination. FIG. 9A shows a case where the pull-up speed of the substrate is 20 cm/min, FIG. 9B shows a case where the pull-up speed of the substrate is 10 cm/min, and FIG. 9C shows a case where the pull-up speed of the substrate is 2 cm/min.

Peaks P1, P2 in the graphs are diffraction peaks from the SU430 substrate. Peak P3 is a diffraction peak from the tantalum oxide and shows that a crystalline tantalum oxide film is formed on the substrate. There is one each of the diffraction peaks, showing that the lamination orientations of crystals are aligned. When the pull-up speed of the substrate is 2 cm/min (FIG. 9C), peak P3 is not observed.

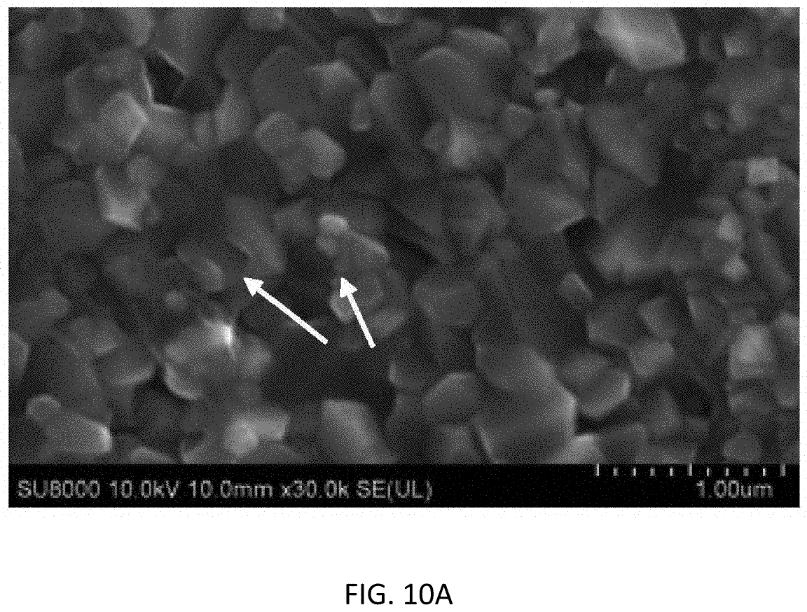

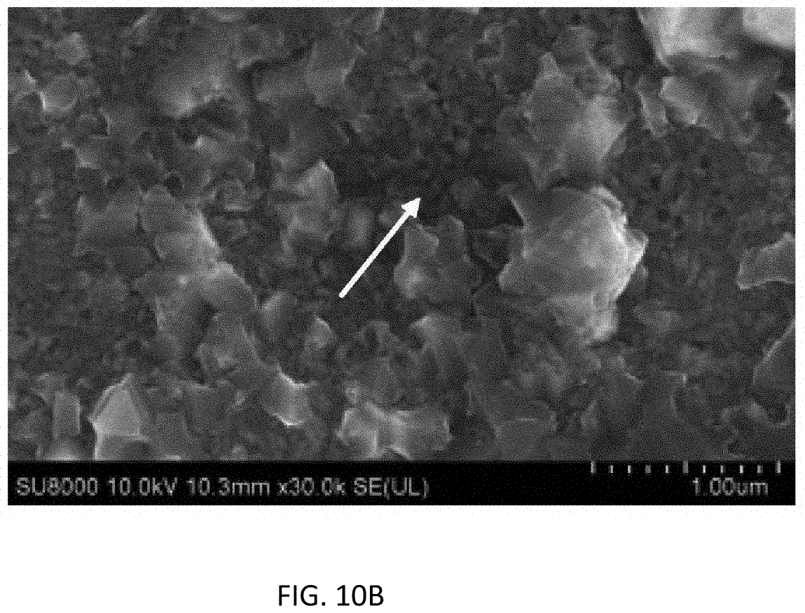

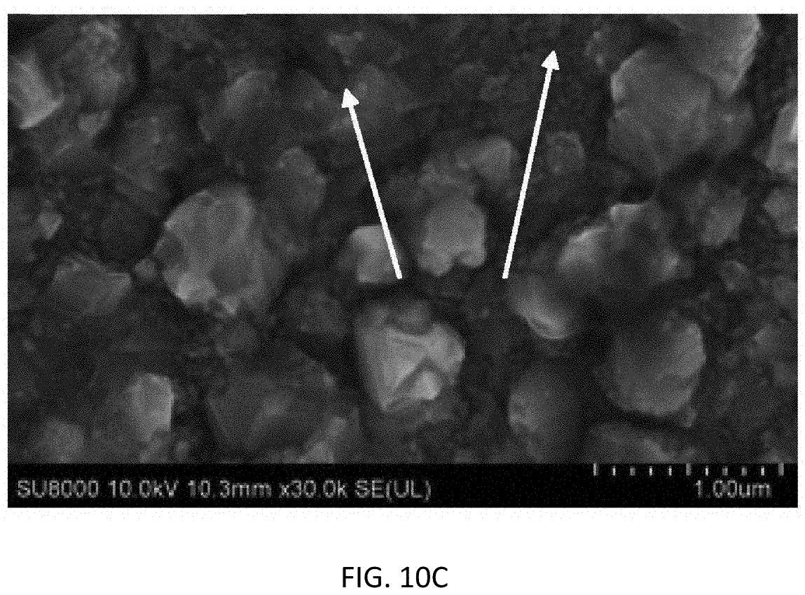

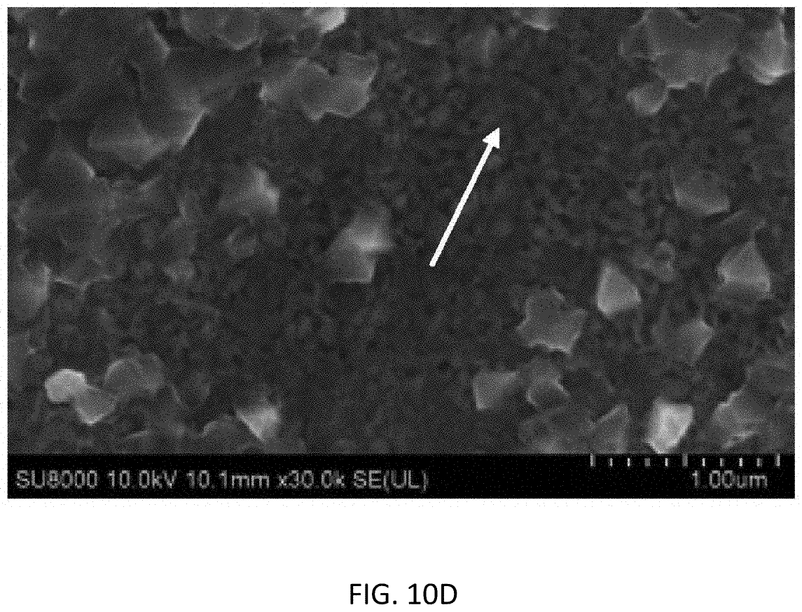

A description will now be given of results of scanning electron microscopic observation of the surface form of samples using the SUS430 substrate. FIG. 10A shows an SEM image of the SUS430 substrate surface without a coating film, FIG. 10B is an SEM image of the SUS430 substrate surface on which a coating film is formed at the pull-up speed of 2 cm/min, FIG. 10C shows an SEM image of the SUS430 substrate surface on which a coating film is formed at the pull-up speed of 10 cm/min, and FIG. 10D is an SEM image of the SUS430 substrate surface on which a coating film is formed at the pull-up speed of 20 cm/min.

The arrow in FIG. 10A shows a crystal precipitated from a stainless substrate. In a region indicated by the arrow in FIG. 10B, minute tantalum oxide particles formed between crystals precipitated from the stainless substrate or on the surface of crystals are observed. Judging from the X-ray diffraction pattern (FIG. 9C) it is considered that the tantalum oxidized film of this thickness is not crystallized and is in an amorphous state.

Meanwhile, in a region indicated by the arrow in FIG. 10C, it is seen that the tantalum oxide film is thicker, and crystals precipitated from the stainless substrate are beginning to be covered by minute tantalum oxide particles. Further, the size of minute tantalum oxide particles is larger than the size of minute tantalum oxide particles shown in FIG. 10B. Further, judging from the X-ray diffraction pattern (FIG. 9B) it is considered that the tantalum oxide film of this thickness is crystallized.

In a region indicated by the arrow in FIG. 10D, the tantalum oxide film is even thicker, and a lot of crystals precipitated from the stainless substrate are beginning to be covered by minute tantalum oxide particles. Further, judging from the X-ray diffraction pattern (FIG. 9A), it is considered that, at this thickness of the tantalum oxide film, a highly crystallized film is formed.

Thus, the method of manufacturing a photocatalytic device according to this embodiment includes heating and calcinating an SUS substrate coated with a tantalum-containing agent at 500° C. or higher, and, preferably, at 700° C. or higher, and, more preferably, at 800° C. or higher, at which the SUS substrate is not melted, to form a photocatalytic layer containing a tantalum oxide on the SUS substrate. According to this manufacturing method, a photocatalytic device provided with a metal layer made of an SUS material and a photocatalytic layer containing a tantalum oxide can be manufactured relatively easily.

Thus, by using an SUS substrate, which is not melted at a relatively high temperature, calcination at a high temperature is made possible, and a photocatalytic layer containing a tantalum oxide and having an excellent crystalline nature can be formed. This can improve the efficiency of decomposition of organic substances, bacteria, etc. by the photocatalytic device. By using an SUS substrate, the cost of the photocatalytic device can be reduced as compared with the case of suing tantalum or titanium in the substrate. Further, using an SUS substrate makes it possible to work the photocatalytic device in a variety of shapes.

While there are a variety of types of SUS substrates, what is required is that the substrate is not softened when calcinated at a desired temperature, at which the crystalline nature of the tantalum oxide as a coating film is improved. SUSs are largely categorized into austenitic series and ferric series. In the case of ferric stainless such as SUS430, a difference in coefficient of thermal expansion from the oxide film is small so that exfoliation is difficult to occur. Therefore, ferric SUS is favorable for use in the substrate as a metal layer of the embodiments. More specifically, ferric SUSs are exemplified by SUS405, SUS410L, SUS429, SUS430, SUS430LX, SUS436L, SUS436JIL, SUS445JI, SUS445J2, SUS444, SUS447J1, SUSXM27, etc.

While ferric stainless is favorable in that the oxide film does not exfoliate easily, an austenitic SUS substrate can be used when an oxide film is not formed on the substrate.

The present invention has been described with reference to the embodiments but is not limited to the embodiments described above. Appropriate combinations or replacements of the features of the illustrated examples are also encompassed by the present invention. Further, the embodiments may be modified by way of combinations, rearranging of the processing sequence, design changes, etc., based on the knowledge of a skilled person, and such modifications are also within the scope of the present invention.

What is claimed is:

1. A photocatalytic device comprising:
a metal layer; and
a photocatalytic layer provided on the metal layer and containing a tantalum oxide, wherein
in the photocatalytic layer, a slit or an opening is formed to expose a portion of the metal layer.

2. The photocatalytic device according to claim 1, wherein
the metal layer contains tantalum.

3. The photocatalytic device according to claim 2, wherein
the photocatalyst layer is produced by anode oxidation of a portion of the metal layer.

4. The photocatalytic device according to claim 1, wherein
the metal layer is made of an SUS (Steel Use Stainless) material.

5. The photocatalytic device according to claim 1, wherein
the photocatalytic layer contains a crystalized tantalum oxide.

6. The photocatalytic device according to claim 1, wherein
a width of the slit or an inner diameter of the opening is within a range of 1-30 μm.

7. The photocatalytic device according to claim 1, wherein
the photocatalytic layer is configured such that a plurality of cells are arranged to sandwich the slit.

8. The photocatalytic device according to claim 1, wherein
a photocatalytic reaction is induced in the photocatalytic layer by ultraviolet light having a peak wavelength in a range of 250-300 nm.

9. The photocatalytic device according to claim 8, wherein
the metal layer is configured such that a positive voltage is applied while the photocatalytic layer is irradiated with an ultraviolet light.

10. A photocatalytic device comprising:
a metal layer made of a ferric SUS material; and
a photocatalytic layer provided on the metal layer and containing a tantalum oxide.

11. A method of manufacturing a photocatalytic device comprising:
heating and calcinating an SUS substrate coated with a tantalum-containing agent in an oxygen-containing atmosphere at 700° C. or above, at which the SUS substrate is not melted, to form a photocatalytic layer containing a tantalum oxide on the SUS substrate.
